(19) United States
(12) Patent Application Publication
(10) Pub. No.: US 2024/0074920 A1
(43) Pub. Date: Mar. 7, 2024

(54) BLADDER CONTROL ARTICLE FOR LEAKAGE CESSATION

(71) Applicant: Shanique T. Cartwright, National Harbor, MD (US)

(72) Inventor: Shanique T. Cartwright, National Harbor, MD (US)

(21) Appl. No.: 17/903,777

(22) Filed: Sep. 6, 2022

(51) Int. Cl. A61F 13/472; A61F 13/15; A61F 13/34; A61F 13/511; A61F 13/534

(52) U.S. Cl. CPC A61F 13/47209; A61F 13/15699; A61F 13/15723; A61F 13/15747; A61F 13/34; A61F 13/47236; A61F 13/51108; A61F 13/53436; A61F 2013/15121; A61F 2013/15365; A61F 2013/15406

(57) ABSTRACT

Disposable absorbent articles suitable for providing bladder leakage cessation for females that experience urinary incontinence may comprise a primary sheet, a secondary sheet, and an absorbent sheet disposed between the primary and secondary sheet. The four-sided primary and secondary sheets each may include four edges, whereby each edge having a selvedge. The absorbent sheet may further include a plurality of macrofolds folded in a counter direction to the primary sheet length and the secondary sheet length. The selvedge edges of the primary sheet and the secondary sheet may couple together around the absorbent sheet to form a pillow package. With the proper placement of the pillow package, bladder leakage can be stopped and mitigated. The article can exhibit an article length and width from about 38 mm to about 50 mm.

### BACKGROUND

As a natural detoxifying process, urine removes the toxins from the human body. Particularly, cellular metabolism generates by-products that are rich in nitrogen, which must be cleared from the blood stream. Trouble arises when a person cannot control the flow of urine, where urine leaks out involuntarily (i.e. urine incontinence). There are three major categories of urinary incontinence: detrusor instability (DI), [also referred to as urge incontinence (UI)]; stress incontinence (SI); and overflow incontinence. DI is urinary leakage due to spontaneous and uninhibited detrusor contractions occurring before the bladder is completely full. Accompanying these contractions is an extremely strong need to urinate. SI is classified as a failure to store urine. In this type of urinary dysfunction, leakage occurs during times of abdominal pressure or stress, such as coughing, sneezing, laughing, bending or lifting heavy objects. The frequency and severity of such urine loss can increase as the muscles and tissues, particularly those near the urethro-vaginal myofascial area, grow weaker. Overflow incontinence is the involuntary loss of urine associated with an over-distended bladder, resulting in frequent to constant leaking of urine without detrusor contractions. Under this condition, the bladder is able to sore urine; yet, bladder emptying is impaired. Individuals with overflow incontinence will typically retain large amounts of urine within the bladder after voiding.

Urinary incontinence is often the primary reason why individuals move to institutions. The annual costs of caring for both ambulatory and institutionalized persons with incontinence, including indirect costs (e.g. the treatment of injuries resulting from falls and complications such as skin breakdown) is estimated to be several billion dollars. Unfortunately, women make up a disproportionally large group that are impacted by urinary incontinence, especially older women and women who have given birth to one or more children, and older women. Many are diagnosed with mixed incontinence, such as a combination of stress incontinence and urge incontinence.

To help those suffering with urinary incontinence, the user may wear a bladder “control” pad. Although bladder control pads absorb large amounts of fluids, skin rashes and embarrassing smells still persist. Furthermore, most bladder control products do not “control” the flow of urine from the bladder, they merely collect a portion of the fluids excreted from the bladder. They do not stop the muscles associated with the bladder to stop the flow of urine.

Additionally, those who suffer from urinary incontinence can turn to bladder control medication as a remedy. Although bladder control drugs may inhibit the production and flow of urine, the side effects can range from annoying to lethal personal injuries and consequences. In the alternative, some surgical procedures exist that allege to correct urinary incontinence. Yet, these types of surgery can be extremely costly, while at the same time providing no guarantee of bladder leakage cessation.

It is within this context that the embodiments arise.

### SUMMARY

Embodiments of an absorbent article for bladder leakage cessation and method for manufacturing the pledget pillow package is provided. It should be appreciated that the present embodiment can be implemented in numerous ways, such as a process, an apparatus, a system, a device, or a method. Several inventive embodiments are described below.

In some embodiments, an absorbent article for bladder leakage cessation is provided. The absorbent article may include a primary sheet having a length, a width and a thickness, wherein the primary sheet includes a body-facing surface and a interior-facing surface. In some embodiments, the primary sheet may include four edges, each having a selvedge. The absorbent article may also include a secondary sheet having a length, a width, and a thickness, wherein the secondary sheet has a body-facing surface and interior-facing surface. In some embodiments, the secondary sheet may include four edges, each having a selvedge. The absorbent article may also include an absorbent sheet (having a length and a width) disposed between the primary sheet interior-facing surface and the secondary sheet interior-facing surface. In some embodiments, the absorbent sheet may include four edges. The length of the absorbent sheet may be greater than the length of the primary and secondary sheet, such that the absorbent sheet having a plurality of macrofolds can be folded in a counter direction to the primary sheet length and the secondary sheet length. To form a pillow package, the selvedge edges of the primary sheet and the secondary sheet may couple together around and encasing the absorbent sheet to form the pillow package. In some embodiments, the pillow package may be spherical in shape for insertion behind the female user clitoris above the urethra to curve the urethra upwards and close the urethra, such that when the labium minora and the labium majora close around the clitoris behind the user's undergarments, the pressure holds the pillow package stable behind the clitoris.

In some embodiments, a pledget for bladder control of a urethra of a female user having labium minora and labium majora is provided. The pledget may include a primary pad having a length, a width and a thickness, wherein the primary pad includes a body-facing surface and a interior-facing surface. In some embodiments, the primary pad may include four edges, each having a selvedge. The pledget may also include a secondary pad having a length, a width, and a thickness, wherein the secondary pad has a body-facing surface and interior-facing surface. In some embodiments, the secondary pad may include four edges, each having a selvedge. The pledget may also include an absorbent pad (having a length and a width) disposed between the primary pad interior-facing surface and the secondary pad interior-facing surface. In some embodiments, the absorbent pad may include four edges. The length of the absorbent pad may be greater than the length of the primary and secondary pad, such that the absorbent pad having a plurality of macrofolds can be folded in a counter direction to the primary pad length and the secondary pad length. To form a pillow package, the selvedge edges of the primary pad and the secondary pad may couple together around and encasing the absorbent pad to form the pillow package. In some embodiments, the pillow package may be compressed into a self-sustaining spherical shape for insertion behind the female user clitoris above the urethra to curve the urethra upwards and close the urethra, such that when the labium minora and the labium majora close around the clitoris, the pressure holds the pillow package stable behind the clitoris.

In some embodiments, method for manufacturing pledget pillow package for bladder control is provided. In an action, the method may include an embossing step of passing a continuous tissue ply paper between an embossing roll and a backing roll making a pair with the embossing roll. The method of manufacturing the pledget pillow package may include a cutting step of cutting the embossed continuous tissue ply paper. The method may further include a folding step of generating macrofolds of an absorbent sheet. The method may further include an attaching step of applying the folded absorbent sheet to a first cut portion of the embossed continuous tissue ply paper. Moreover, the method may include an encasing step of joining a second cut portion of the embossed continuous tissue ply paper to the first cut portion of the embossed continuous tissue ply paper to form the pillow package. In some embodiments, the method may include a coupling step of joining a withdrawal string to one of the cut portion of the embossed continuous tissue ply paper for ease of user removal.

Other aspects and advantages of the embodiments will become apparent from the following detailed description taken in conjunction with the accompanying drawings which illustrate, by way of example, the principles of the described embodiments.

### BRIEF DESCRIPTION OF THE DRAWINGS

The described embodiments and the advantages thereof may best be understood by reference to the following description taken in conjunction with the accompanying drawings. These drawings in no way limit any changes in form and detail that may be made to the described embodiments by one so skilled in the art without departing from the spirit and scope of the described embodiments.

FIG. 1a is a perspective view of an absorbent article for bladder leakage cessation having an embossed tissue ply, in accordance with some embodiments.

FIG. 1B is a cross-sectional view of the absorbent article for bladder leakage cessation of FIG. 1a, in accordance with some embodiments.

FIG. 1c is a deconstructed view of the absorbent article for bladder leakage cessation of FIG. 1B, in accordance with some embodiments.

FIG. 1d is a top view of the absorbent sheet for insertion between a primary and secondary sheet of the absorbent article for bladder leakage cessation of FIG. 1c, in accordance with some embodiments.

FIG. 2a is a perspective view of an absorbent article for bladder leakage cessation having a macro-folded tissue ply, in accordance with some embodiments.

FIG. 2b is a cross-sectional view of a second embodiment of the absorbent article for bladder leakage cessation of FIG. 2a, in accordance with some embodiments.

FIG. 3 is a perspective view of an absorbent article for bladder leakage cessation having an embossed tissue ply with a withdrawal string attached thereto, in accordance with some embodiments.

FIG. 4a is a cross-sectional view of an embossed primary and secondary tissue ply of FIG. 1a, in accordance with some embodiments.

FIG. 4b is a cross-sectional view of the primary and secondary tissue ply having macrofolds of FIG. 2a, in accordance with some embodiments.

FIG. 5a is a front view of the female anatomy having a weakened bladder muscle, in accordance with some embodiments.

FIG. 5b is front view of the female anatomy of FIG. 5a having the innovative absorbent article for bladder leakage cessation of FIG. 1a, positioned behind the user clitoris above the urethra, in accordance with some embodiments.

FIG. 5c is the cross-sectional view of the female anatomy having a weakened bladder muscle of FIG. 5a, in accordance with some embodiments.

FIG. 5d is the cross-sectional view of the female anatomy of FIG. 5b having the innovative absorbent article for bladder leakage cessation of FIG. 1a, positioned behind the user clitoris above the urethra, in accordance with some embodiments.

FIG. 6 is a block diagram showing a method of manufacturing the absorbent article for bladder leakage cessation and pledget pillow package for bladder control in accordance with some embodiments.

### DETAILED DESCRIPTION

The following embodiments describe an absorbent article for bladder leakage cessation and method for manufacturing the pledget pillow package. It can be appreciated by one skilled in the art, that the embodiments may be practiced without some or all of these specific details. In other instances, well known process operations have not been described in detail in order not to unnecessarily obscure the embodiments.

In some embodiments, an absorbent article for bladder leakage cessation is provided. The absorbent article may include a primary sheet having a length, a width and a thickness, wherein the primary sheet includes a body-facing surface and a interior-facing surface. In some embodiments, the primary sheet may include four edges, each having a selvedge. The absorbent article may also include a secondary sheet having a length, a width, and a thickness, wherein the secondary sheet has a body-facing surface and interior-facing surface. In some embodiments, the secondary sheet may include four edges, each having a selvedge. The absorbent article may also include an absorbent sheet (having a length and a width) disposed between the primary sheet interior-facing surface and the secondary sheet interior-facing surface. In some embodiments, the absorbent sheet may include four edges. The length of the absorbent sheet may be greater than the length of the primary and secondary sheet, such that the absorbent sheet having a plurality of macrofolds can be folded in a counter direction to the primary sheet length and the secondary sheet length. To form a pillow package, the selvedge edges of the primary sheet and the secondary sheet may couple together around and encasing the absorbent sheet to form the pillow package. In some embodiments, the pillow package may be spherical in shape for insertion behind the female user clitoris above the urethra to curve the urethra upwards and close the urethra, such that when the labium minora and the labium majora close around the clitoris behind the user's undergarments, the pressure holds the pillow package stable behind the clitoris.

In some embodiments, the primary and the secondary sheet may include a first tissue ply coupled to a second tissue ply. The first tissue ply may include a first machine direction length (MD Length), wherein the first tissue ply serves as the interior-facing surface of the primary sheet. The second tissue ply, having a second MD Length, may include a plurality of macrofolds. In some embodiments, each of the plurality of macrofolds may comprise a different shape. Some of the plurality of macrofolds may possess differing macrofold segment lengths. In some embodiments, the second tissue ply may be embossed. The coupling of the first tissue ply and the second tissue ply may occur at each valley of each macrofold of the second tissue, wherein the second tissue ply serves as the body-facing surface of the primary sheet. In some embodiments, the first and the second tissue ply may have a basis weight from about 10 to about 60 gsm and a sheet bulk greater than about 5 cc/g.

The article may be a pledget for bladder control of a urethra of a female user having labium minora and labium majora. The difference between the absorbent article and the pledget being the material composition of each, wherein the absorbent article is made of tissue ply and the pledget being made of thin absorbent pads. Particularly, in some embodiments, a pledget for bladder control of a urethra of a female user may include a primary pad having a length, a width and a thickness, wherein the primary pad includes a body-facing surface and a interior-facing surface. In some embodiments, the primary pad may include four edges, each having a selvedge. The pledget may also include a secondary pad having a length, a width, and a thickness, wherein the secondary pad has a body-facing surface and interior-facing surface. In some embodiments, the secondary pad may include four edges, each having a selvedge. The pledget may also include an absorbent pad (having a length and a width) disposed between the primary pad interior-facing surface and the secondary pad interior-facing surface. In some embodiments, the absorbent sheet may include four edges. The length of the absorbent pad may be greater than the length of the primary and secondary pad, such that the absorbent pad having a plurality of macrofolds can be folded in a counter direction to the primary pad length and the secondary pad length. To form a pillow package, the selvedge edges of the primary pad and the secondary pad may couple together around and encasing the absorbent pad to form the pillow package. In some embodiments, the pillow package may be compressed into a self-sustaining spherical shape for insertion behind the female user clitoris above the urethra to curve the urethra upwards and close the urethra, such that when the labium minora and the labium majora close around the clitoris, the pressure holds the pillow package stable behind the clitoris. In some embodiments, a withdrawal string may be coupled to one of the selvedge edges of the primary pad and the secondary pad for ease of user removal.

Advantageously, the innovative design of the absorbent article for bladder leakage cessation and/or the pledget for bladder control of a urethra of a female user curtails most, if not all bladder leakage. The novel design and novel application alters the shape and opening of the urethra, such that the urethra is tilted upwards and closed. This upwards, closed position completely stops most if not all bladder leakage. The pressure from the closure of the labium minora and the labium majora around the clitoris and the absorbent article disclosed herein, keeps the absorbent article in place even during activities that exert pressure on the abdomen, such as laughing, coughing, exercising, and the like. Moreover, since urinary incontinence is often the primary reason why individuals move to institutions, many more people will be able to live on their own unassisted without the major costs of health care facilities. Since the annual costs of caring for both ambulatory and institutionalized persons with incontinence, including indirect costs (e.g. the treatment of injuries resulting from falls and complications such as skin breakdown), is estimated to be several billion dollars, this innovative concept can save billions of dollars. Additionally, the mental health of multiplied millions of people can be enhanced by the absorbent article for bladder leakage cessation and/or the pledget for bladder control as described herein, whereby users may no longer experience the embarrassment and shame often associated with urinary incontinence.

In the following description, numerous details are set forth. It will be apparent, however, to one skilled in the art, that the present invention may be practiced without these specific details. In some instances, well-known structures and devices are shown in block diagram form, rather than in detail, in order to avoid obscuring the present invention.

Reference in the description to “one embodiment” or “an embodiment” means that a particular feature, structure, or characteristic described in connection with the embodiment is included in at least one embodiment of the invention. The phrase “in one embodiment” located in various places in this description does not necessarily refer to the same embodiment. Like reference numbers signify like elements throughout the description of the figures.

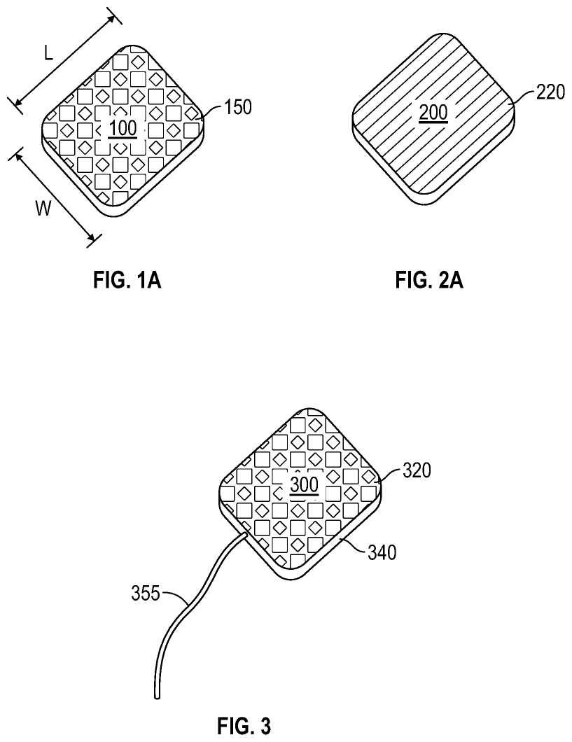

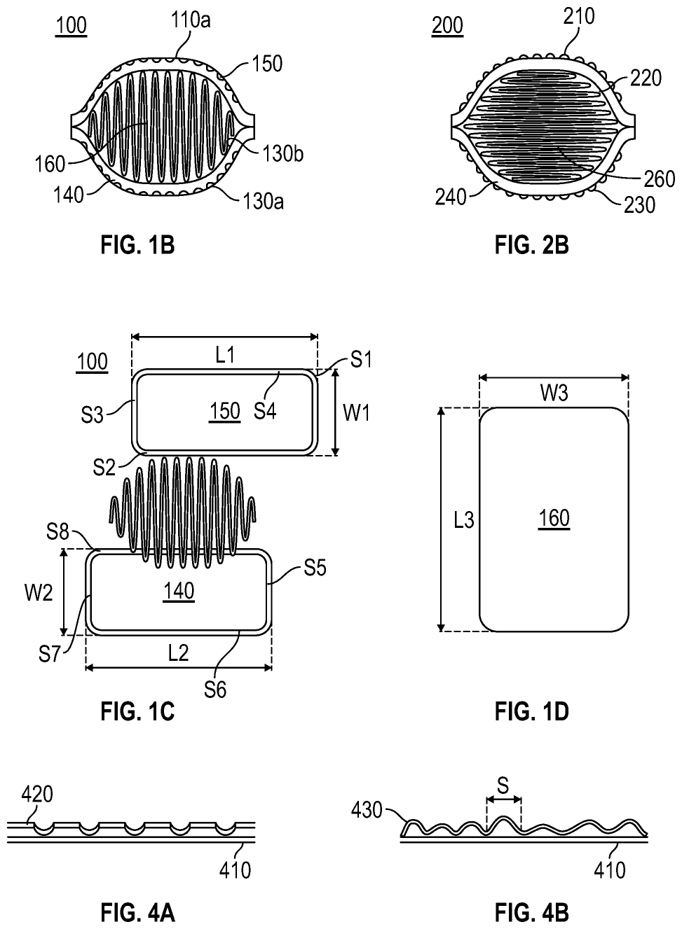

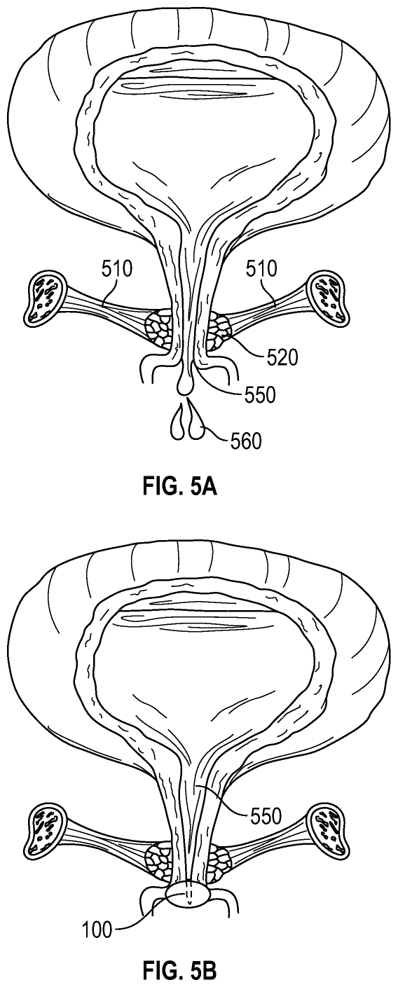

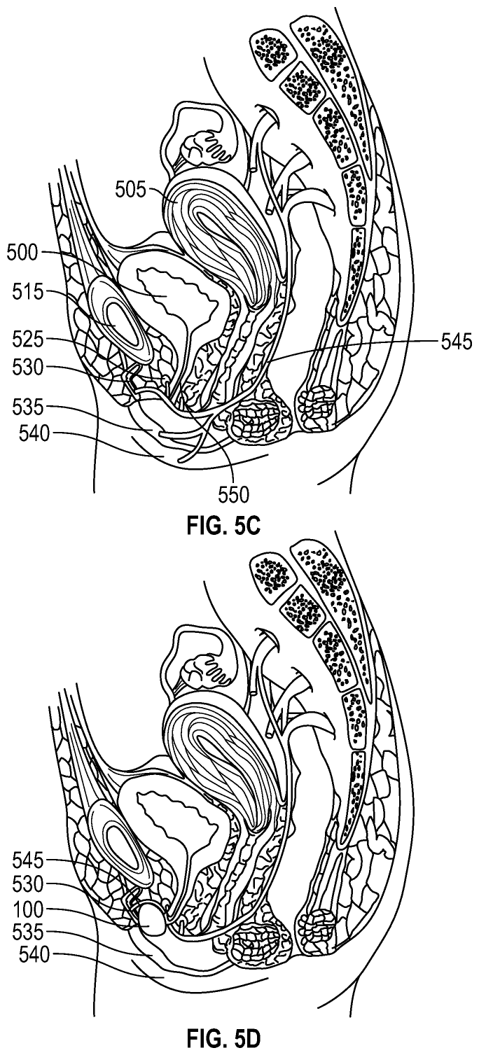

Referring to FIG. 1a, a perspective view of an absorbent article for bladder leakage cessation, in accordance with some embodiments is shown. The absorbent article 100 for bladder leakage cessation is may include a primary sheet 150 having a length (L₁), a width (W₁), and a thickness (t), wherein the primary sheet includes a body-facing surface 110a and a interior-facing surface 110b (as shown in FIGS. 1B and 1c). Particularly, referring to FIG. 1B, a cross-sectional view of the absorbent article for bladder leakage cessation of FIG. 1a, in accordance with some embodiments is shown. Further details are shown in FIG. 1c, where a deconstructed view of the absorbent article for bladder leakage cessation of FIG. 1B, is shown. In some embodiments, the primary sheet 150 may include four edges, each having a selvedge (S₁, S₂, S₃, and S₄). The absorbent article may also include a secondary sheet 140 having a length (L₂), a width (W₂), and a thickness (t₂), wherein the secondary sheet 140 has a body-facing surface 130a and interior-facing surface 130b. As shown in FIG. 1a, the body-facing surface 110a and 130a may be embossed. In some embodiments, the secondary sheet may 140 include four edges, each having a selvedge (S₅, S₆, S₇, and S₈). The absorbent article 100 may also include an absorbent sheet 160 (having a length (L₃), a width (W₃) disposed between the primary sheet 150 interior-facing surface 110b and the secondary sheet 140 interior-facing surface 130b. Referring to FIG. 1d, a top view of the absorbent sheet for insertion between a primary and secondary sheet of the absorbent article for bladder leakage cessation of FIG. 1c is shown. In some embodiments, the absorbent sheet 160 may include four edges. The length L₃ of the absorbent sheet 160 may be greater than the lengths (L₁, L₂) of the primary and secondary sheet (150, 140), such that the absorbent sheet 160 having a plurality of macrofolds can be folded in a counter-direction to the direction of the primary sheet length (L₁) and the secondary sheet length (L₂). To form a pillow package 100, the selvedge edges of the primary sheet (S₁, S₂, S₃, S₄) and the secondary sheet (S₅, S₆, S₇, S₈) may couple together around and encasing the absorbent sheet 160 to form the pillow package 100. In some embodiments, the pillow package 100 may be spherical in shape for insertion behind the female user clitoris above the urethra to curve the urethra upwards and close the urethra, such that when the labium minora and the labium majora close around the clitoris, the pressure holds the pillow package stable behind the clitoris (to be explained in further detail with respect to FIGS. 5a-5d).

In operation, when the innovative absorbent article 100 is placed slightly in front of the urethra and tucked behind the clitoris (as detailed in FIGS. 5c and 5d), the urethra is curved slightly upward and closes. When the user needs to relieve her bladder, she can simply sit on the toilet and naturally the labium minora and a labium majora will open (as detailed in FIGS. 5c and 5d). The user can release the holding of the urine by simply releasing the pelvic muscles to begin to urinate; and the flow of urine will force the absorbent article 100 into the toilet. Since the absorbent article 100 can be made from materials ordinarily used to make tissue ply for toilet paper, the absorbent article 100 can be conveniently disposed of by flushing it down the toilet. Advantageously, the absorbent article 100 is kept in place by the pressure of the labium minora and the labium majora behind the user's undergarments, even during activities that exert pressure on the abdomen, such as laughing, coughing, exercising, and the like.

Referring to FIG. 2a, is a perspective view of an absorbent article 200 for bladder leakage cessation having a macro-folded tissue ply, in accordance with some embodiments. Particularly, referring to FIG. 2b, a cross-sectional view of a second embodiment of the absorbent article 200 for bladder leakage cessation of FIG. 2a in accordance with some embodiments is shown. In some embodiments, the primary sheet 220, having a length (L₁), a width (W₁), and a thickness (t₁), may include four edges, each having a selvedge. The absorbent article may also include a secondary sheet 240 having a length (L₂), a width (W₂), and a thickness (t₂), wherein the secondary sheet 140 has a body-facing surface 230a and interior-facing surface 130b. As shown in FIG. 1a, the body-facing surface 210a and 230a may include macro-folded tissue ply as shown in FIG. 2a. In some embodiments, the secondary sheet may 240 include four edges, each having a selvedge. The absorbent article 200 may also include an absorbent sheet 260 (having a length (L₃), a width (W₃) disposed between the primary sheet 220 interior-facing surface 210b and the secondary sheet 240 interior-facing surface 230b. In some embodiments, the absorbent sheet 260 may include four edges. The length L₃ of the absorbent sheet 260 may be greater than the lengths (L₁, L₂) of the primary and secondary sheet (220, 240), such that the absorbent sheet 260 having a plurality of macrofolds can be folded in the same direction of the primary sheet length (L₁) and the secondary sheet length (L₂) as shown in FIG. 2b. To form a pillow package 200, the selvedge edges of the primary sheet (220) and the secondary sheet (240) may couple together around and encasing the absorbent sheet 260 to form the pillow package 200. In some embodiments, the pillow package 200 may be spherical in shape for insertion behind the female user clitoris above the urethra to curve the urethra upwards and close the urethra, such that when the labium minora and the labium majora close around the clitoris, the pressure holds the pillow package stable behind the clitoris (to be explained in further detail with respect to FIGS. 5a-5d).

Referring to FIG. 3, a perspective view of an absorbent article for bladder leakage cessation having an embossed tissue ply with a withdrawal string attached thereto, in accordance with some embodiments. Particularly, the absorbent article 300 for bladder leakage cessation may include a withdrawal string 355 coupled to the pillow package formed by coupling primary sheet 320 and secondary sheet 340. In some embodiments the withdrawal string 355 may be coupled to the primary sheet 320 prior to the coupling to the primary sheet 320 to the secondary sheet 340. Particularly, the withdrawal string 355 may be coupled to one of the selvedge edges of the primary sheet 320. In the alternative, the withdrawal string 355 may be coupled to the secondary sheet 340, prior to the coupling of the primary and the secondary sheets (320, 340).

In operation, when the innovative absorbent article 100 is placed slightly in front of the urethra and tucked behind the clitoris (as detailed in FIGS. 5c and 5d), the urethra is curved slightly upward and closes. Advantageously, the absorbent article 100 is kept in place by the pressure of the labium minora and the labium majora behind the user's undergarments, even during activities that exert pressure on the abdomen, such as laughing, coughing, exercising, and the like. When the user needs to relieve her bladder, she can simply sit on the toilet and pull the withdrawal string 355 before releasing the muscles that hold the urine in the bladder. Since the absorbent article 100 can be made from materials ordinarily used to make tissue ply for toilet paper, the absorbent article 100 can be conveniently disposed of by flushing the article 100 down the toilet.

Referring to FIG. 4a, a cross-sectional view of an embossed primary and secondary tissue ply of FIG. 1a, in accordance with some embodiments is shown. Particularly, the primary and the secondary sheet may include a first tissue ply 410 coupled to a second tissue ply 420. The first tissue ply 410 may include a first machine direction length (MD Length), wherein the first tissue ply 410 serves as the interior-facing surface (130b) of the primary sheet 150. In some embodiments, the second tissue ply 420 may be embossed, where it serves as the body-facing surface (130a) of the primary sheet 150. In some embodiments, the second tissue ply 430, having a second MD Length, may include a plurality of macrofolds as shown in FIG. 4b. Particularly, referring to FIG. 4b, a cross-sectional view of the primary and secondary tissue ply having macrofolds of FIG. 2a, in accordance with some embodiments is displayed. In some embodiments, each of the plurality of macrofolds may comprise differing shapes (e.g. small, medium, large folds and the like). Some of the plurality of macrofolds may possess differing macrofold segment lengths (s), as shown in FIG. 4b. The coupling of the first tissue ply 410 and the second tissue ply 430 may occur at each valley of each macrofold of the second tissue 430, wherein the second tissue ply 430 serves as the body-facing surface (210) of the primary sheet 220. In some embodiments, the first and the second tissue ply (410, 420, 430) may have a basis weight from about 10 to about 60 gsm and a sheet bulk greater than about 5 cc/g.

As to the tissue ply, a known raw material pulp can be used for the (continuous) crepe paper according to some embodiments. For example, wood pulp, recycled pulp, and the like may be utilized. In particular, the preferred raw material pulp is a combination of NBKP (Nadelholz Bleached Kraft Pulp) and LBKP (Laubhoulz Bleached Kraft Pulp). Although, recycled pulp may be added, a mixture of only NBKP and LBKP of virgin pulp is more preferable in terms of the favorable hand feel and the like. In this case, the mixture ratio is NBKP:LBKP=10:90 to 50:50, in particular desirably NBKP:LBKP=40:60.

A paper raw material such as raw material pulp becomes a crepe paper through, for example, a known paper manufacturing process, via a wire part, a press part, a dryer part, a size press, a calender part, and the like. In the paper manufacturing, appropriate chemicals e.g., a dispersing agent, a pH adjuster such as caustic soda, an antifoaming agent, an antiseptic agent, and a slime control agent may be added.

As for paper quality of the crepe paper, the basis weight is preferably 12.0 to 30.0 g/m² in one-ply. When the basis weight is less than 12.0 g/m², the thickness to the touch feeling is lost and paper breakage exists after the embossing the tissue ply. In contrast, when the basis weight is more than 30.0 g/m², the quality of the paper is degraded, due to surface roughness or decreased water disintegration property of the tissue ply.

In some embodiments, paper thickness (per one tissue ply) of the continuous crepe paper may be 100 to 330 μm in two sheets according to measurement by a dial thickness gauge (Peacock thickness gage). When the paper thickness is smaller than 100 μm, the thickness to the touch feeling is lost and the paper is likely to stretch, which makes it difficult to locate emboss positions accurately. Conversely, when the paper thickness is larger than 330 μm, the paper can be rough, which is undesirable.

Tensile strength measured can be measured according to the state of the products. That is, one-ply products are measured in the state of the one-ply, while two-ply products are measured in the state of the two-ply. For example, in a case of the two-ply products, it is preferable that the tensile strength (in the length direction) is 320 to 500 cN/25 mm while the tensile strength (in the width direction) is 140 to 250 cN/25 mm. The tensile strengths of the continuous crepe paper, which do not reach the tensile strength in the length direction or width direction, can be ineffective, due to paper breakage during the embossing phase of manufacturing the paper.

When manufacturing the embossed tissue ply, continuous crepe paper can be pressed by embossing convex portions, which are formed on the peripheral surface of a convex embossing roll (not shown) of a tissue ply manufacturing machine (not shown), whereby the tissue ply is passed between rolls having the desired embossing pattern.

Referring to FIG. 5a, a front view of the female anatomy having a weakened bladder muscle, in accordance with some embodiments is illustrated. As shown, a bladder 500, having a bladder neck 502, may include a urethra 550. A pair of weak pelvic muscles 510 may couple to a pair of urethral sphincter 520 for controlling the start and stop of the flow of urine. In particular, during the malfunctioning of bladder control, the weak pelvic muscles 510 will not be strong enough to apply pressure upon the urethral sphincter 520 which contracts.

In operation, when the innovative absorbent article 100 is placed slightly in front of the urethra and tucked behind the clitoris (as detailed in FIGS. 5c and 5d), the urethra is curved slightly upward and closes. Particularly, referring to FIG. 5b, a front view of the female anatomy of FIG. 5a having the innovative absorbent article for bladder leakage cessation positioned behind the user clitoris above the urethra, in accordance with some embodiments is illustrated. When the absorbent article 100 is placed slightly in front of the urethra 550, the urethra curves forward and closes as illustrated by the dotted lines.

Referring to FIG. 5c, the cross-sectional view of the female anatomy having a weakened bladder muscle of FIG. 5a, in accordance with some embodiments is illustrated. In particular, the female anatomy comprises a uterus 505 (having a vaginal canal 555), a bladder 500 (having a urethra 550 and urethral sphincter 520 coupled to constrict the flow of urine), a pelvic bone 515, a clitoris 530, a labium minora 535, and a labium majora 540. As shown, there is a spatial distance between the clitoris 530 and the urethra 550. Within in this spatial distance, the absorbent article 100 can be placed slightly in front of the urethra 550 and tucked behind the clitoris 530 (as detailed in FIG. 5d). Particularly, referring to FIG. 5d, the cross-sectional view of the female anatomy of FIG. 5a having the innovative absorbent article for bladder leakage cessation positioned behind the user clitoris above the urethra, in accordance with some embodiments is illustrated. As shown, when the absorbent article 100 is placed slightly in front of the urethra 550, the urethra 550 curves forward and closes as illustrated.

Ordinarily, the urinary sphincter muscle 520, which is located at the upper end of the urethra 550, adjacent to the bladder 500, seals off the passing of urine from the bladder 500 to the urethra 550, when it has a generally round or circular cross-sectional configuration. Support of the proximal urethra 550 elevates muscle 520 above the pelvic floor and subjects it to increases in intra-abdominal pressure, thus allowing compression and maintenance of continence. However, when this passageway becomes distorted into an elliptical or oval cross-sectional configuration, the sphincter muscle 520 cannot close properly, therefore, the tendency for involuntary urine loss increases. SI can be caused by damage to the proximal urethra 550 through trauma, radiation, sacral spinal cord lesions, prior surgeries, estrogen deficiency, or a congenital weakness. Additionally, neuromuscular damage from pregnancy, childbirth, and pelvic surgery is a common cause of SI.

In operation, when the innovative absorbent article 100 is placed slightly in front of the urethra 550 and tucked behind the clitoris 530 (as detailed in FIGS. 5c and 5d), the urethra 550 is curves slightly upward and closes. The curve and closure of the urethra stops the urine from leaking out. The body-facing surface of the absorbent article 100 absorbs any liquid present and is soft enough to be reshaped to the contour of the female user's body. The pressure from the closure of the labium minora 535 and the labium majora 540, closing around the clitoris 530, the holds the pillow package 100 stable behind the clitoris 530, while at the same time curving the urethra 550 upwards and closed. When the user needs to relieve her bladder, she can sit on the toilet and naturally the labium minora 535 and a labium majora 540 will open. The user can release the holding of the urine by simply releasing the pelvic muscles 510 to begin to urinate; and the flow of urine will force the absorbent article 100 into the toilet. Since the absorbent article 100 can be made from materials ordinarily used to make tissue ply for toilet paper, the absorbent article 100 can be conveniently disposed of by flushing it down the toilet.

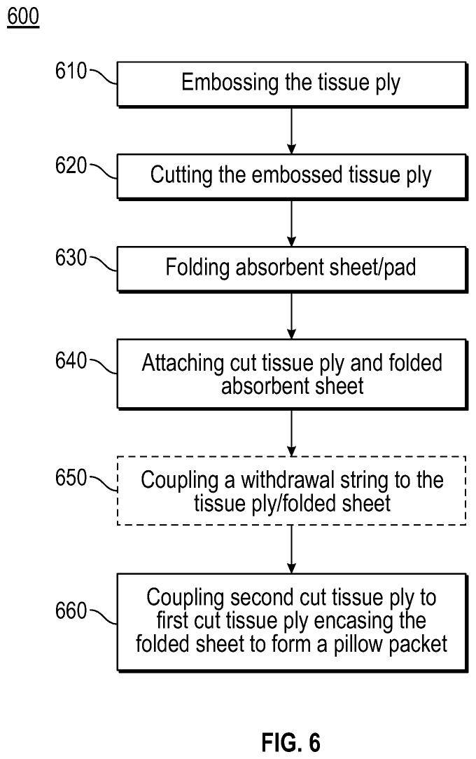

Referring to FIG. 6, a block diagram showing a method of manufacturing the absorbent article for bladder leakage cessation and pledget pillow package for bladder control in accordance with some embodiments is displayed. Particularly, the method 600 for manufacturing pledget pillow package for bladder control is provided. In an action 610, the method may include an embossing step of passing a continuous tissue ply paper between an embossing roll and a backing roll making a pair with the embossing roll. The method of manufacturing the pledget pillow package may include a cutting step of cutting the embossed continuous tissue ply paper, in an action 620. The method may further include a folding step of generating macrofolds of an absorbent sheet, in an action 630. In an action, 640, the method may further include an attaching step of applying the folded absorbent sheet to a first cut portion of the embossed continuous tissue ply paper. In some embodiments, the method may include a coupling step of joining a withdrawal string to one of the cut portion of the embossed continuous tissue ply paper for ease of user removal, in an action 650. Moreover, the method may include an encasing step of joining a second cut portion of the embossed continuous tissue ply paper to the first cut portion of the embossed continuous tissue ply paper to form the pillow package, in an action 660.

In the above description, numerous details are set forth. It will be apparent, however, to one skilled in the art, that the present invention may be practiced without these specific details. In some instances, well-known structures and devices are shown in block diagram form, rather than in detail, in order to avoid obscuring the present invention.

It is to be understood that the above description is intended to be illustrative, and not restrictive. Many other embodiments will be apparent to those of skill in the art upon reading and understanding the above description. Although the present invention has been described with reference to specific exemplary embodiments, it will be recognized that the invention is not limited to the embodiments described, but can be practiced with modification and alteration within the spirit and scope of the appended claims. Accordingly, the specification and drawings are to be regarded in an illustrative sense rather than a restrictive sense. The scope of the invention should, therefore, be determined with reference to the appended claims, along with the full scope of equivalents to which such claims are entitled.

It should be understood that although the terms first, second, etc. may be used herein to describe various steps or calculations, these steps or calculations should not be limited by these terms. These terms are only used to distinguish one step or calculation from another. For example, a first calculation could be termed a second calculation, and, similarly, a second step could be termed a first step, without departing from the scope of this disclosure. As used herein, the term “and/or” and the “I” symbol includes any and all combinations of one or more of the associated listed items. As used herein, the singular forms “a”, “an” and “the” are intended to include the plural forms as well, unless the context clearly indicates otherwise. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. Therefore, the terminology used herein is for the purpose of describing particular embodiments only and is not intended to be limiting.

Although the method operations were described in a specific order, it should be understood that other operations may be performed in between described operations, described operations may be adjusted so that they occur at slightly different times or the described operations may be distributed in a system which allows the occurrence of the processing operations at various intervals associated with the processing.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the embodiments and its practical applications, to thereby enable others skilled in the art to best utilize the embodiments and various modifications as may be suited to the particular use contemplated. Accordingly, the present embodiments are to be considered as illustrative and not restrictive, and the invention is not to be limited to the details given herein, but may be modified within the scope and equivalents of the appended claims.

What is claimed is:

1. An absorbent article for bladder leakage cessation, comprising:
a primary sheet having a body-facing surface and a interior-facing surface, the primary sheet having four edges each with a selvedge, the primary sheet having a first length and a first width;
a secondary sheet having a body-facing surface and interior-facing surface, the secondary sheet having four edges each with a selvedge, the secondary sheet having a second length and a second width; and
an absorbent sheet disposed between the primary sheet interior-facing surface and the secondary sheet interior-facing surface, the absorbent sheet having four edges, the absorbent sheet having a third length and a third width;
wherein the absorbent sheet having a plurality of macrofolds folded in a counter direction to the primary sheet length and the secondary sheet length; and
wherein the selvedge edges of the primary sheet and the secondary sheet couple together around the absorbent sheet to form a pillow package.

2. The absorbent article of claim 1, wherein the primary sheet comprises,
a first tissue ply having a first machine direction length (MD Length), wherein the first tissue ply is the interior-facing surface of the primary sheet; and
a second tissue ply, having a plurality of macrofolds and a second MD Length, coupled to the first tissue ply, wherein the second tissue ply couples to the first tissue ply at each valley of each macrofold of the second tissue, wherein the second tissue ply is the body-facing surface of the primary sheet;
wherein the first MD Length is substantially less than the second MD Length.

3. The absorbent article of claim 2, wherein the first tissue ply having basis weight from about 10 to about 60 gsm and a sheet bulk greater than about 5 cc/g.

4. The absorbent article of claim 2, wherein the second tissue ply having basis weight from about 10 to about 60 gsm and a sheet bulk greater than about 5 cc/g.

5. The absorbent article of claim 2, wherein the second tissue ply is embossed.

6. The absorbent article of claim 2, wherein each of the plurality of macrofolds have a different shape.

7. The absorbent article of claim 2, wherein each of the plurality of macrofolds have a different macrofold segment length.

8. The absorbent article of claim 1, wherein the secondary sheet comprises,
a third tissue ply having a third machine direction length (MD Length), wherein the third tissue ply is the interior-facing surface of the secondary sheet; and
a fourth tissue ply, having a plurality of macrofolds and a fourth MD Length, coupled to the third tissue ply, wherein the fourth tissue ply couples to the third tissue ply at each valley of each macrofold of the fourth tissue, wherein the fourth tissue ply is the body-facing surface of the secondary sheet;
wherein the third MD Length is substantially equal to the fourth MD Length.

9. The article of claim 8, wherein the third tissue ply having basis weight from about 10 to about 60 gsm and a sheet bulk greater than about 5 cc/g.

10. The article of claim 8, wherein the fourth tissue ply having basis weight from about 10 to about 60 gsm and a sheet bulk greater than about 5 cc/g.

11. The article of claim 8, wherein the fourth tissue ply is embossed.

12. The article of claim 8, wherein each of the plurality of macrofolds have a different shape.

13. The article of claim 8, wherein each of the plurality of macrofolds have a different macrofold segment length.

14. A pledget for bladder control of a urethra of a female user having labium minora and labium majora, comprising:
a primary pad having a length, a width and a thickness, the primary pad having a body-facing surface and a interior-facing surface, the primary pad having four edges each with a selvedge;
a secondary pad having a length, a width, and a thickness, the secondary pad having a body-facing surface and interior-facing surface, the secondary pad having four edges each with a selvedge; and
an absorbent pad disposed between the primary pad interior-facing surface and the secondary pad interior-facing surface, the absorbent pad having four edges, the absorbent sheet having a length and a width;
wherein the absorbent pad having a plurality of macrofolds folded in a counter direction to the primary pad length and the secondary pad length;
wherein the selvedge edges of the primary pad and the secondary pad couple together around the absorbent pad to form a pillow package;
wherein the pillow package is compressed into a self-sustaining spherical shape for insertion behind the female user clitoris above the urethra to curve the urethra upwards and close the urethra behind the labium minora and the labium majora.

15. The pledget of claim 14, further comprises,
a withdrawal string, coupled to one of the selvedge edges of the primary pad and the secondary pad for ease of user removal.

16. The pledget of claim 14, wherein the length and the width of the primary pad being approximately 38 mm, the primary pad consisting essentially of viscose rayon fiber and having a weight between about 0.94 grams and about 1.78 grams.

17. The pledget of claim 14, wherein the length and the width of the secondary pad being approximately 38 mm, the secondary pad consisting essentially of viscose rayon fiber and having a weight between about 0.76 grams and about 1.46 grams.

18. A method for manufacturing pledget pillow package for bladder control, comprising:
an embossing step of passing a continuous tissue ply paper between an embossing roll and a backing roll making a pair with the embossing roll;
a cutting step of cutting the embossed continuous tissue ply paper;
a folding step of generating macrofolds of an absorbent sheet;
an attaching step of applying the folded absorbent sheet to a first cut portion of the embossed continuous tissue ply paper; and
an encasing step of joining a second cut portion of the embossed continuous tissue ply paper to the first cut portion of the embossed continuous tissue ply paper to form the pillow package.

19. The pledget of claim 18, further comprises,
a coupling step of joining a withdrawal string to one of the cut portion of the embossed continuous tissue ply paper for ease of user removal.

20. The pledget of claim 18, wherein each cut portion of the embossed continuous tissue ply paper having a length and a width being approximately 38 mm.
